(19) United States
(12) Patent Application Publication
(10) Pub. No.: US 2026/0092868 A1
(43) Pub. Date: Apr. 2, 2026

(54) METHOD FOR SCREENING FOR AUTOPHAGY MODULATOR

(71) Applicants: THE ASAN FOUNDATION, Seoul (KR); UNIVERSITY OF ULSAN FOUNDATION FOR INDUSTRY COOPERATION, Ulsan (KR)

(72) Inventors: Chan Gi PACK, Seoul (KR); Jun Ki KIM, Seoul (KR); In Ki KIM, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 19/112,113

(22) PCT Filed: Sep. 11, 2023
(86) PCT No.: PCT/KR2023/013595

§ 371 Date: Jun. 10, 2025

(30) Foreign Application Priority Data
Sep. 16, 2022 (KR) 10-2022-0117375

(51) Int. Cl. G01N 21/64; G01N 21/41; G01N 33/483

(52) U.S. Cl. CPC G01N 21/6486; G01N 21/41; G01N 21/6428; G01N 21/6456; G01N 33/483

(57) ABSTRACT

Provided is a method of screening for an autophagy modulator. According to the screening method of an aspect, the autophagy modulator can be accurately screened with only a single cell. In addition, screening can be performed without using fluorescent labeling, and thus cytotoxicity and photobleaching that occur when fluorescent labeling analysis methods are used can be prevented, and the limitation of having to monitor only fluorescently labeled markers can be improved.

### TECHNICAL FIELD

The present disclosure relates to a method of screening for an autophagy modulator.

### BACKGROUND ART

Autophagy is well known as a process necessary for eliminating unnecessary components occurring within cells and maintaining cellular homeostasis. However, if there is a problem with this autophagic process, aggregates of various biological components such as undegraded intracellular proteins may accumulate, resulting in occurrence of various diseases.

Meanwhile, it has recently been discovered that inhibiting autophagy that is activated in specific cancer cells may be effective in anticancer treatment. Therefore, the development of autophagy modulators that artificially activate or inhibit these cellular processes is becoming increasingly important.

However, currently, the drug development or drug mechanism research of autophagy modulators relies solely on a method in which cells overexpressed by tagging LC3B, which is an representative autophagy biomarker, with a fluorescence protein such as GFP or RFP by a genetic engineering technique are observed with fluorescence microscopy, and this invasive method has many disadvantages, such as cytotoxicity caused by fluorescence during monitoring, reduced reliability of analysis results due to photobleaching of fluorescent molecules, inefficiency in 3D image acquisition time, and monitoring being limited only to fluorescently labeled marker molecules.

### DISCLOSURE OF INVENTION

### Technical Problem

An aspect provides a method of screening for an autophagy modulator, including bringing a biological sample into contact with a candidate substance, measuring the refractive index (RI) distribution of an organelle in the biological sample in contact with the candidate substance, and extracting a physical quantity of the organelle by using the measured RI distribution.

### Solution to Problem

An aspect provides a method of screening for an autophagy modulator, including bringing a biological sample into contact with a candidate substance, measuring the refractive index (RI) distribution of an organelle in the biological sample in contact with the candidate substance; and extracting a physical quantity of the organelle by using the measured RI distribution.

The biological sample may be a tissue or a cell. The cell may be a cell population or a single cell, and the cell population may consist of single cells, include a plurality of homogeneous cells, or include a plurality of heterogeneous cells.

In an embodiment, the cell may be a somatic cell or a cancer cell.

The term “somatic cell” as used herein may refer to any cell other than germ cells. The somatic cell may be, for example, derived from mammals such as humans, horses, sheep, pigs, goats, camels, antelopes, and dogs, or isolated from mammals.

In an embodiment, the somatic cell may be any one or more selected from the group consisting of muscle cells, liver cells, fibroblasts, epithelial cells, neurons, adipocytes, bone cells, blood cells, mucosal cells, and stem cells, but the present disclosure is not limited thereto.

The cancer cell may be a cell from liver cancer, squamous cell carcinoma, uterine cancer, cervical cancer, prostate cancer, head and neck cancer, pancreatic cancer, brain tumor, breast cancer, skin cancer, esophageal cancer, testicular cancer, kidney cancer, colorectal cancer, rectal cancer, gastric cancer, bladder cancer, ovarian cancer, bile duct cancer, or gallbladder cancer, but the present disclosure is not limited thereto.

The term “organelle” as used herein may refer to an organ that constitutes a cell. The organelle may have a size (diameter) in the nanometer or micrometer range, for example, from 0.1 μm to 100 μm, from 0.1 μm to 50 μm, from 0.1 μm to 25 μm, from 0.1 μm to 15 μm, from 0.1 μm to 10 μm, from 0.1 μm to 5 μm, or from 0.1 μm to 3 μm, but the present disclosure is not limited thereto.

In an embodiment, the organelle may be any one or more selected from the group consisting of an endoplasmic reticulum (ER), a vesicle, a lipid droplet, a mitochondrion, a ribosome, a Golgi apparatus, an endosome, a nucleolus, and a lysosome. The ER may be a rough ER (rER) or a smooth ER (sER).

The vesicle may have a structure consisting of a lipid bilayer, and the vesicle may be an autophagosome.

The term “refractive index (RI)” as used herein may refer to an inherent optical physical quantity of a material itself that indicates the degree to which light slows down as it passes through the material. Also, because the RI is inversely proportional to the density of a material, the RI may refer to biological information that reflects the density of a cell or an organelle.

In an embodiment, the measurement of the RI distribution may include the steps of: generating a plurality of two-dimensional holograms by using diffraction interferometer-based quantitative phase imaging (QPI); and measuring three-dimensional RI distribution by analyzing the plurality of two-dimensional holograms.

The QPI may involve measuring three-dimensional RI distribution by measuring, by using an interferometer, a complex optical field that forms the amplitude and phase delay of diffracted and transmitted light as light is diffracted by a biological sample. The complex optical field may be formed and measured at various incident angles, and the three-dimensional RI distribution of the biological sample corresponding to the concentration of each organelle may be measured on the basis of the complex optical field.

In an embodiment, the diffraction interferometer-based QPI may be optical diffraction tomography (ODT).

In an embodiment, the screening method may include a step of generating an image for extracting a physical quantity of an organelle of a cell by using the three-dimensional RI distribution.

The image may be a three-dimensional image. The three-dimensional image may be generated by using three-dimensional RI distribution generated by using a plurality of two-dimensional holograms that are generated from two-dimensional RI distribution of an organelle on the basis of the difference in RI between the organelle and the cytoplasm. By generating the three-dimensional image, the organelle may be three-dimensionally imaged, and the physical quantity of the organelle may be quantified on the basis of the three-dimensional image.

In an embodiment, the physical quantity may be at least one of a volume of the organelle, an RI of the organelle, a density of the organelle, and a number of the organelles.

The term “autophagy” as used herein may refer to a catabolism process for clearance of cellular components including unnecessary or denatured proteins within cells. Therefore, autophagy modulators and autophagy modulation methods may be effectively used in the treatment or prevention of various diseases caused by autophagy modulation abnormalities as described above.

The term “modulation” as used herein may include activation, stimulation or up-regulation, or reduction or down-regulation, or both, of a biological function, and may include modulation in an in vitro state, modulation in an in vivo state, and modulation in an ex vivo state. Therefore, the modulator may include an autophagy activator or enhancer, or an autophagy inhibitor.

In an embodiment, the autophagy modulator may be an autophagy activator or an autophagy inhibitor. The autophagy activator or the autophagy inhibitor may include a drug having the ability to activate or inhibit autophagy, nanoparticles, or nanoparticles encapsulating the drug. The drug having the ability to activate or inhibit autophagy may include thapsigargin, cyclopiazonic acid, tunicamycin, torin, valproic acid, verapamil hydrochloride, carbamazepine, dexamethasone, niclosamide, nimodipine, nitrendipine, rapamycin, or colchicine.

In an embodiment, the screening method may include a step of measuring the physical quantity of the organelle in a cell in contact with the candidate substance over time.

In an embodiment, the screening method may include a step of determining that, when the volume of a vesicle in a cell in contact with the candidate substance increases over time, the candidate substance is an autophagy activator.

In an embodiment, the screening method may include a step of determining that, when the volume of a vesicle in a cell in contact with the candidate substance does not change or decreases over time, the candidate substance is an autophagy inhibitor.

In an embodiment, the screening method may include a step of determining that, when the number of vesicles in a cell in contact with the candidate substance decreases over time, the candidate substance is an autophagy activator.

In an embodiment, the screening method may include a step of determining that, when the number of vesicles in a cell in contact with the candidate substance does not change or increases over time, the candidate substance is an autophagy inhibitor.

In an embodiment, the screening method may include a step of determining that, when the density of a vesicle in a cell in contact with the candidate substance decreases over time, the candidate substance is an autophagy activator.

In an embodiment, the screening method may include a step of determining that, when the density of a vesicle in a cell in contact with the candidate substance does not change or increases over time, the candidate substance is an autophagy inhibitor.

In an embodiment, the screening method may include comparing the extracted physical quantity of the organelle with the physical quantity of an organelle in a biological sample not in contact with the candidate substance. In this regard, the physical quantity of the organelle in the biological sample in contact with the candidate substance may be a physical quantity extracted immediately after being brought into contact with the candidate substance.

In an embodiment, the screening method may include a step of determining that, when the volume of a vesicle in a cell immediately after being brought into contact with the candidate substance increases compared with the volume of a vesicle in a cell not in contact with the candidate substance, the candidate substance is an autophagy activator.

In an embodiment, the screening method may include a step of determining that, when the volume of a vesicle in a cell immediately after being brought into contact with the candidate substance does not change or decreases compared with the volume of a vesicle in a cell not in contact with the candidate substance, the candidate substance is an autophagy inhibitor.

In an embodiment, the screening method may include a step of determining that, when the number of vesicles in a cell immediately after being brought into contact with the candidate substance increases compared with the number of vesicles in a cell not in contact with the candidate substance, the candidate substance is an autophagy activator.

In an embodiment, the screening method may include a step of determining that, when the number of vesicles in a cell immediately after being brought into contact with the candidate substance does not change or decreases compared with the number of vesicles in a cell not in contact with the candidate substance, the candidate substance is an autophagy inhibitor.

In an embodiment, the screening method may include a step of determining that, when the density of a vesicle in a cell immediately after being brought into contact with the candidate substance increases compared with the density of a vesicle in a cell not in contact with the candidate substance, the candidate substance is an autophagy activator.

In an embodiment, the screening method may include a step of determining that, when the density of a vesicle in a cell immediately after being brought into contact with the candidate substance does not change or decreases compared with the density of a vesicle in a cell not in contact with the candidate substance, the candidate substance is an autophagy inhibitor.

The vesicle may include other organelles or organelle components. For example, the vesicle may include an ER or an ER component, and the ER or the ER component may be encapsulated in the vesicle.

In an embodiment, the screening method may include a step of measuring the expression of a biomarker in the organelle. The measurement of the expression of a biomarker in the organelle may be performed by immunofluorescence (IF), western blotting, or fluorescence imaging, but the present disclosure is not limited thereto. The measurement of the expression of a biomarker in the organelle may be performed simultaneously or at different times with the diffraction interferometer-based QPI. Accordingly, the reliability of the diffraction interferometer-based QPI may be improved.

In an embodiment, the biomarker in the organelle may be a biomarker for autophagy. The biomarker for autophagy may be microtubule-associated protein 1 light chain 3 alpha (LC3). In addition, a biomarker for ER-specific autophagy may be translocation protein SEC62 (SEC62), reticulon-3 (RTN3), recticulophagy regulator 1 (FAM134B), or cell-cycle progression gene 1 (CCPG1), but the present disclosure is not limited thereto.

In an embodiment, the measurement of the expression of a biomarker in the organelle may include a step of treating a cell with a fluorescent tracker. The fluorescent tracker may be an organelle-specific fluorescent tracker, and a change in organelle may be specifically confirmed through the fluorescent tracker. The fluorescent tracker may be, for example, ER-specific ER-tracker, lysosome-specific lysosome-tracker, or mitochondria-specific mito-tracker, but the present disclosure is not limited thereto.

In an embodiment, the autophagy modulator may be used for the prevention or treatment of cancer, a neurodegenerative disease, an autoimmune disease, a cardiovascular disease, a metabolic disease, or a hereditary muscle disease.

### Advantageous Effects of Invention

According to a screening method of an aspect, an autophagy modulator can be accurately screened with only a single cell. In addition, screening can be performed without using fluorescent labeling, and thus cytotoxicity and photobleaching that occur when fluorescent labeling analysis methods are used can be prevented, and the limitation of having to monitor only fluorescently labeled markers can be improved.

### BRIEF DESCRIPTION OF DRAWINGS

FIG. 1 illustrates optical diffraction tomography (ODT) images showing a process of high-density vesicles being produced and disappearing over time after SNU475 cells were treated with thapsigargin.

FIG. 2 illustrates graphs showing changes in the refractive index and radius of vesicles in SNU475 cells treated with thapsigargin over time:

FIG. 2A is a graph showing the change in the refractive index of vesicles in SNU475 cells treated with thapsigargin over time; and FIG. 2B is a graph showing the change in the radius of vesicles in SNU475 cells treated with thapsigargin over time.

FIG. 3 is a graph showing a change in the total number of vesicles in SNU475 cells treated with thapsigargin and a change in volume per vesicle over time.

FIG. 4 illustrates images and graphs showing classification according to the number and refractive index of vesicles produced in SNU475 cells treated with thapsigargin, and graphs showing changes in the refractive indices of organelles before and after SNU475 cells were treated with thapsigargin:

FIG. 4A illustrates images showing comparison in the number and refractive index of vesicles produced in SNU475 cells treated with thapsigargin; FIG. 4B is a graph showing the refractive index of vesicles produced in SNU475 cells treated with thapsigargin; and FIG. 4C is a graph showing the refractive indices of organelles in SNU475 cells treated with thapsigargin.

FIG. 5 illustrates images showing changes in vesicles and lysosomes after SNU475 cells were treated with thapsigargin (10 μM), and then stained with ER-tracker or lysosome-tracker:

FIG. 5A illustrates observation images of a change in the endoplasmic reticulum upon treatment with thapsigargin after being stained with ER-tracker; and

FIG. 5B illustrates observation images of a change in lysosomes upon treatment with thapsigargin after being stained with lysosome-tracker.

FIG. 6 illustrates observation images of a change in mitochondria upon treatment with thapsigargin after being stained with mito-tracker.

FIG. 7 is a graph showing the results of confirming whether CCPG1, FAM134B, RTN3, and SEC62, which are ER-phagy receptors, were activated upon treatment with thapsigargin.

FIG. 8 illustrates ODT images obtained by observing a change in vesicles after SNU475 cells were treated with cyclopiazonic acid (CPA) or tunicamycin (TC):

FIG. 8A illustrates ODT images showing the change in vesicles upon treatment with CPA; and FIG. 8B illustrates ODT images showing the change in vesicles upon treatment with TC.

### MODE FOR THE INVENTION

Hereinafter, the present disclosure will be described in further detail with reference to the following examples. However, these examples are provided for illustrative purposes only and are not intended to limit the scope of the present disclosure.

### Example 1. Confirmation of Changes in Vesicles in SNU475 Cells Treated with Thapsigargin (TG)

To confirm changes in vesicles upon treatment with an autophagy modulator, SNU475 cells, which are a human hepatoma cell line, were treated with 10 μM thapsigargin, which is an autophagy activator, and then the changes in vesicles in the cells were examined.

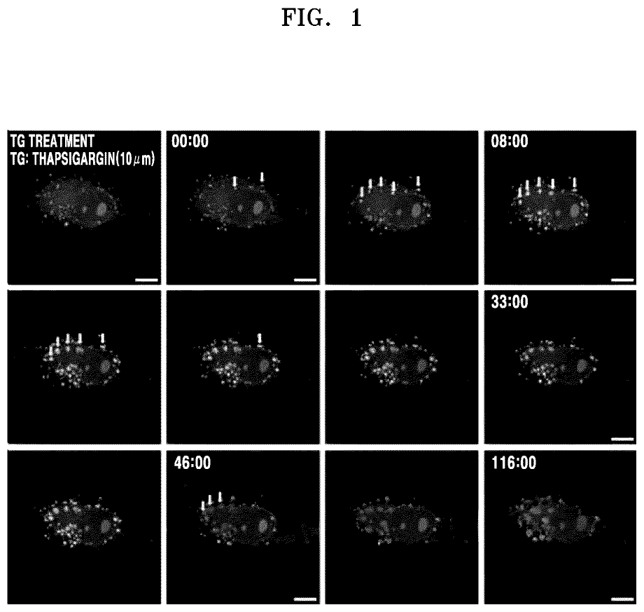

More specifically, a SNU475 cell line was prepared in a TomoDish and the SNU475 cells were treated with 10 μM thapsigargin, and then optical diffraction tomography (ODT) images were generated by using HT-2H fluorescence holotomography (Tomocube). The results thereof are shown in FIG. 1.

FIG. 1 illustrates time-dependent ODT images showing changes in vesicles occurring in SNU475 cells after being treated with thapsigargin.

As illustrated in FIG. 1, it was confirmed that high-density vesicles were generated upon treatment with thapsigargin, which is an autophagy activator.

### Example 2. Confirmation of Changes in Refractive Index and Radius of Vesicles in SNU475 Single Cell Treated with Thapsigargin

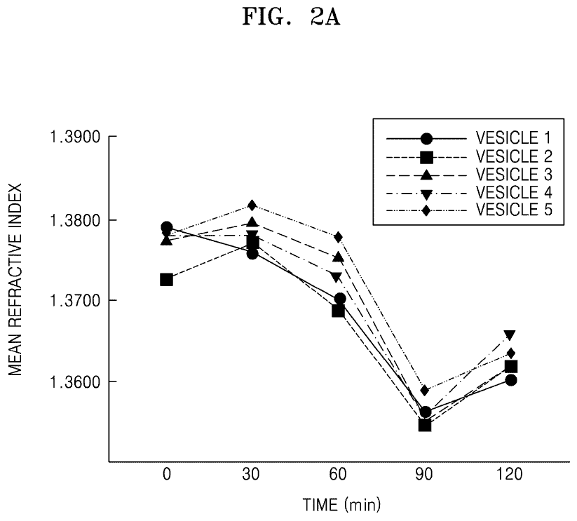

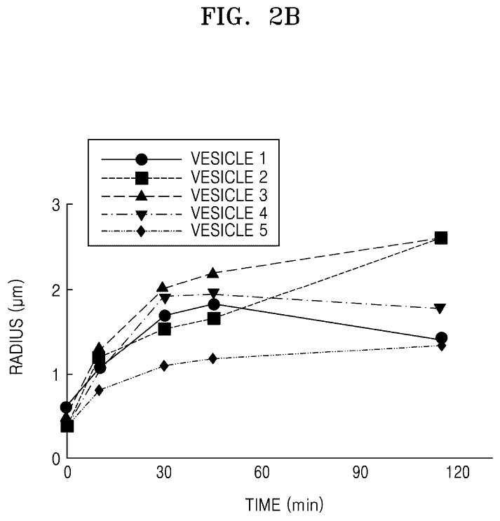

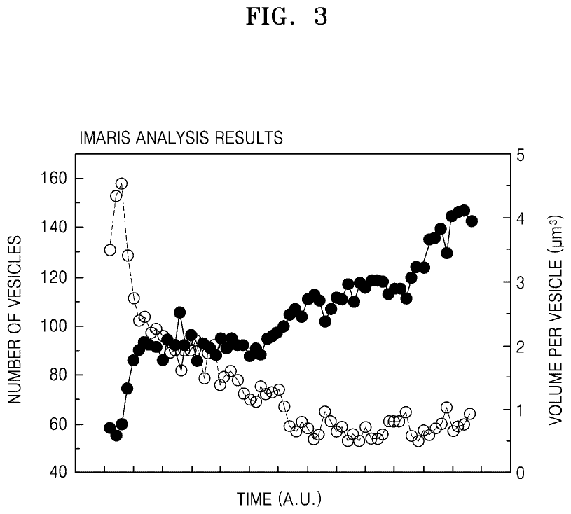

SNU475 cells were treated with 10 μM thapsigargin to induce ER stress, and then changes in the refractive index and radius of vesicles produced in the cells were measured on the basis of images confirmed in Example 1, and the results thereof are shown in FIG. 2. In addition, SNU475 cells were treated with 10 μM thapsigargin, and the number and volume of vesicles in the SNU475 cells treated with thapsigargin were observed over time for 2 hours, and the results thereof are shown in FIG. 3.

FIG. 2 illustrates graphs showing changes in the refractive index and radius of vesicles in SNU475 cells treated with thapsigargin over time:

FIG. 2A is a graph showing the change in the refractive index of vesicles in SNU475 cells treated with thapsigargin over time; and FIG. 2B is a graph showing the change in the radius of vesicles in SNU475 cells treated with thapsigargin over time.

FIG. 3 is a graph showing a change in the total number of vesicles in SNU475 cells treated with thapsigargin and a change in volume per vesicle over time.

As illustrated in FIG. 2, it was confirmed that the refractive index of vesicles produced upon treatment with thapsigargin, which is an autophagy activator, decreased over time. It was also confirmed that the radius of vesicles produced upon treatment with thapsigargin increased over time.

As illustrated in FIG. 3, it was confirmed that the total number of vesicles produced upon treatment with thapsigargin, which is an autophagy activator, decreased over time due to fusion between the vesicles. It was also confirmed that the volume of vesicles produced upon treatment with thapsigargin increased over time.

These results indicate that, upon treatment of a cell with a specific autophagy modulator candidate substance, it may be determined that, when the density of vesicles in the cell decreases, the radius or volume of vesicles in the cell increases, or the total number of vesicles in the cell decreases, the specific autophagy modulator candidate substance is an autophagy activator.

### Example 3. Confirmation of Statistical (or Systematic) Changes in Refractive Index and Radius of Vesicles and Organelles in SNU475 Cells Treated with Thapsigargin

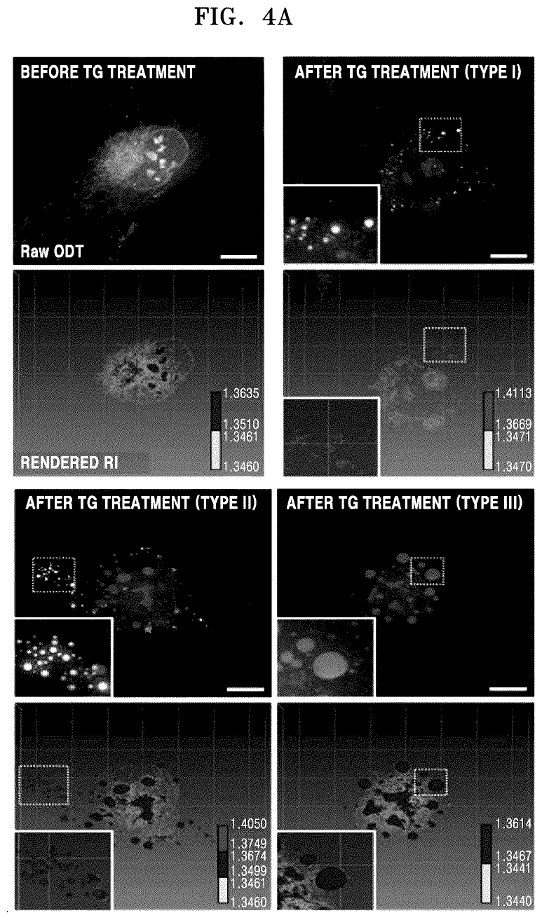

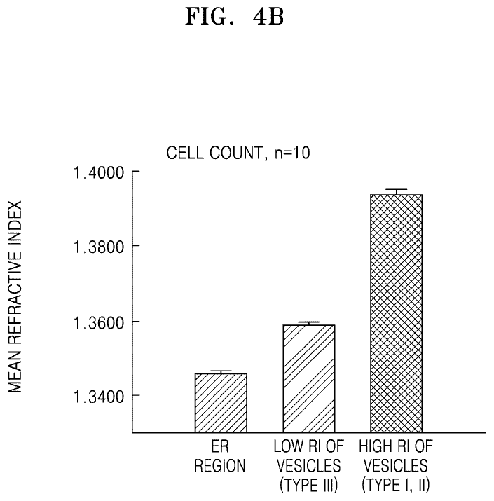

To confirm statistical changes in the density and refractive index of vesicles and organelles upon treatment with thapsigargin, the densities and refractive indices of cells before and after SNU475 cells were treated with thapsigargin (10 μM) were compared. In addition, to confirm changes in the densities of organelles upon treatment with thapsigargin, the refractive indices of a nucleolus (No), an endoplasmic reticulum (ER), a cytosol (Cyt), and a plasma membrane (PM) were measured. The results thereof are shown in FIG. 4.

FIG. 4 illustrates images and graphs showing classification according to the number and refractive index of vesicles produced in SNU475 cells treated with thapsigargin, and graphs showing changes in the refractive indices of organelles before and after SNU475 cells were treated with thapsigargin:

FIG. 4A illustrates images showing comparison in the number and refractive index of vesicles produced in SNU475 cells treated with thapsigargin; FIG. 4B is a graph showing the refractive index of vesicles produced in SNU475 cells treated with thapsigargin; and FIG. 4C is a graph showing the refractive indices of organelles in SNU475 cells treated with thapsigargin.

As illustrated in FIG. 4, it was confirmed that the greater the number of vesicles, the higher the refractive index thereof. That is, it was confirmed that the number and refractive index of vesicles are proportional to each other. It was also confirmed that vesicles induced by thapsigargin had a higher density than the endoplasmic reticulum, and the density of the endoplasmic reticulum decreased upon treatment with thapsigargin. From these results, it was confirmed that ER-phagy was induced upon treatment with thapsigargin, resulting in production of vesicles including high-density ER components.

### Example 4. Confirmation of ER-Specific Changes by using Fluorescent Tracker

4.1. Image Analysis using ER-Tracker and Lysosome-Tracker

Changes in the endoplasmic reticulum and lysosomes were measured using a confocal microscope and fluorescent trackers. As the fluorescent trackers, ER-tracker, which enables the specific observation of only the endoplasmic reticulum, and lysosome-tracker, which enables the specific observation of only lysosomes, were used.

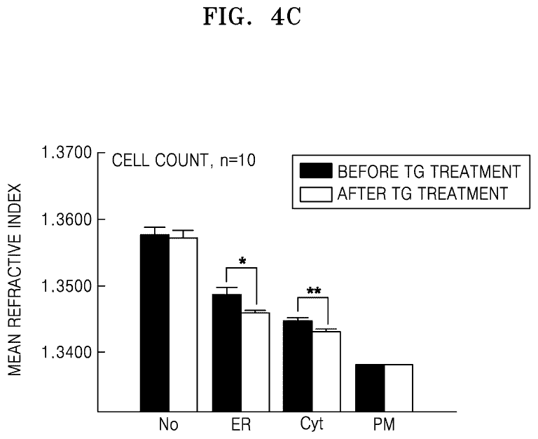

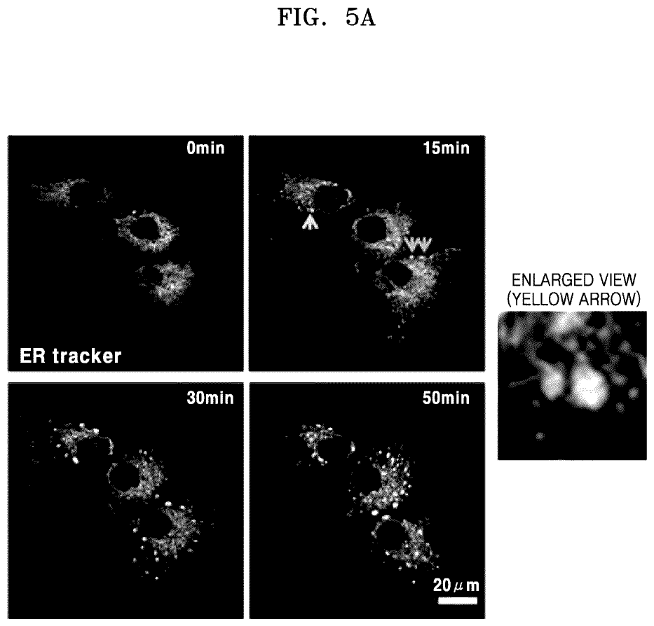

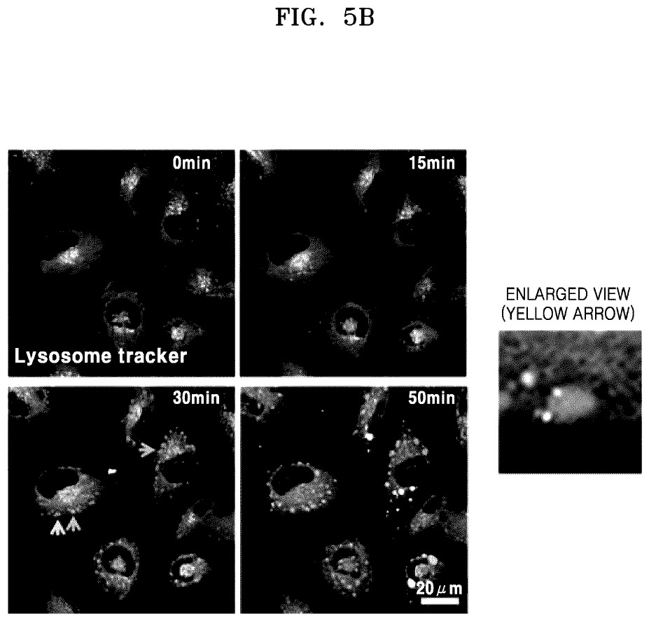

Specifically, SNU475 cells were treated with thapsigargin (10 μM) and then stained with ER-tracker or lysosome-tracker, the kinetics of the endoplasmic reticulum and lyosomes were analyzed by super-resolution microscopy (Airyscan), and the results thereof are shown in FIG. 5.

FIG. 5 illustrates images showing changes in vesicles and lysosomes after SNU475 cells were treated with thapsigargin (10 μM), and then stained with ER-tracker or lysosome-tracker:

FIG. 5A illustrates observation images of a change in the endoplasmic reticulum upon thapsigargin treatment after being stained with ER-tracker, wherein, in FIG. 5A, an arrow indicates a portion where vesicles having ER components start to be produced; and FIG. 5B illustrates observation images of a change in lysosomes upon treatment with thapsigargin after being stained with lysosome-tracker, wherein, in FIG. 5B, an arrow indicates a portion where large lysosomes are produced with a time difference (about 15 minutes) relative to the ER.

As illustrated in FIG. 5, it was confirmed that vesicles and lysosomes that contain ER components were produced in SNU475 cells upon treatment with thapsigargin. This is the same result as that confirmed through ODT. That is, this indicates that autophagy modulators can be effectively screened by confirming the physical quantities of organelles through ODT.

Additionally, changes in the endoplasmic reticulum and lysosomes in SNU475 cells treated with thapsigargin were analyzed using an ODT setup that can link three-dimensional ODT imaging and two-dimensional fluorescence. As a result, it was confirmed that, after treatment with thapsigargin, the structure of high-density vesicles produced in the cytoplasm specifically colocalized with the endoplasmic reticulum and lysosomes. It was also confirmed that vesicles were produced in the ER region, followed by fusion with lysosomes.

These results indicate that, upon treatment with thapsigargin, vesicles in cells include endoplasmic reticulum components, and vesicles including endoplasmic reticulum components are degraded by fusion with lysosomes, resulting in changes in the density of vesicles.

4.2. Image Analysis using Mito-Tracker

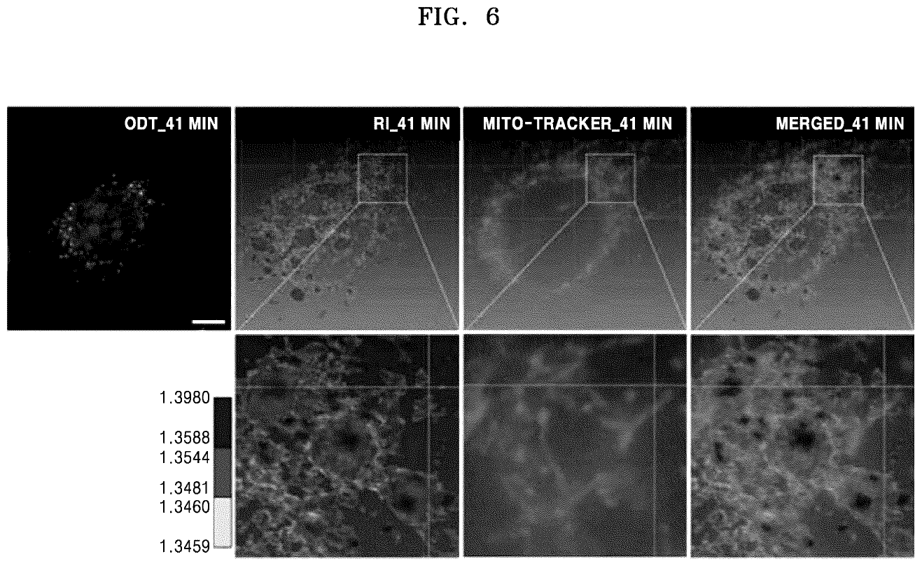

To confirm that high-density vesicles produced upon treatment with thapsigargin are specific to the endoplasmic reticulum, the kinetics of mitochondria were analyzed in the same manner as in 4.1 above by using mito-tracker, which is a mitochondria-specific tracker, and the results thereof are shown in FIG. 6.

FIG. 6 illustrates observation images of a change in mitochondria upon treatment with thapsigargin after being stained with mito-tracker.

As illustrated in FIG. 6, it was confirmed that high-density vesicles produced upon treatment with thapsigargin did not include mitochondrial components.

These results indicate that high-density vesicles induced upon treatment with thapsigargin are specific to the endoplasmic reticulum.

4.3. Measurement of colocalization of ER-phagy receptors

To measure the activation levels of an autophagy marker and an ER-phagy marker after treatment with thapsigargin, the colocalization of ER-phagy receptors was measured.

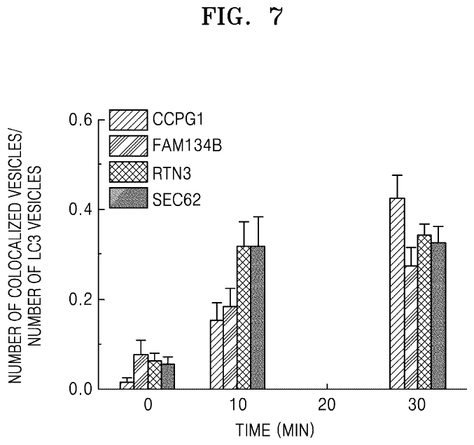

More specifically, after treatment with thapsigargin (10 μM), the activation of CCPG1, FAM134B, RTN3, and SEC62 was examined by immunofluorescence (IF) analysis, and then the results thereof are shown in FIG. 7.

FIG. 7 is a graph showing the results of confirming whether CCPG1, FAM134B, RTN3, and SEC62, which are ER-phagy receptors, were activated upon treatment with thapsigargin.

As illustrated in FIG. 7, it was confirmed that, upon treatment with thapsigargin, ER-phagy receptors CCPG1, FAM134B, RTN3, and SEC62 were activated through colocalization with LC3.

These results indicate that high-density vesicles observed through ODT after treatment with thapsigargin are produced by ER-phagy.

### Example 5. Confirmation of Changes in Refractive Index and Radius of Vesicles in SNU475 Cells Treated with Cyclopiazonic Acid (CPA) or Tunicamycin (TC)

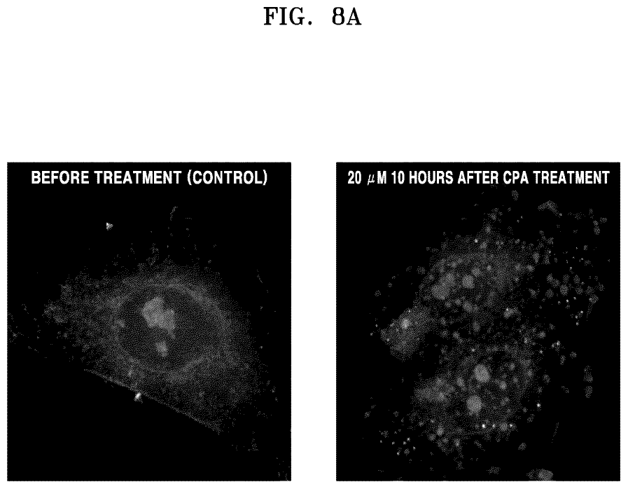

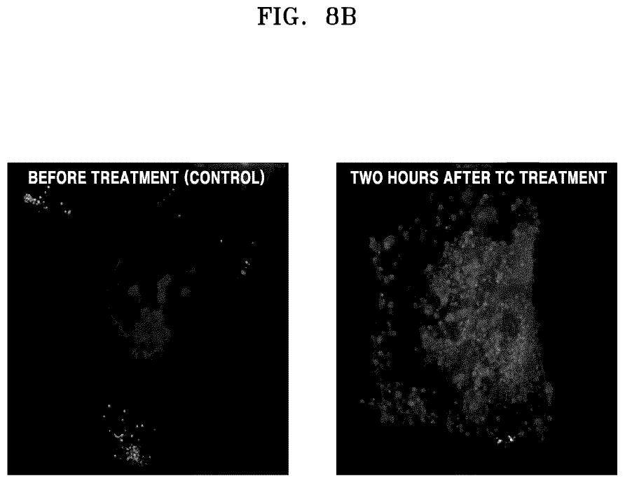

To confirm whether the same results as the case of thapsigargin can be obtained upon treatment with autophagy modulators other than thapsigargin, SNU475 cells were treated with each of CPA (20 μM) and TC (60 ug/mL) to induce ER stress, and then changes in vesicles produced in the cells were observed using HT-2H fluorescence holotomography (Tomocube). For observation, optical diffraction tomography (ODT) images 10 hours after treatment with CPA and ODT images 2 hours after treatment with TC were generated, and the results thereof are shown in FIG. 8.

FIG. 8 illustrates ODT images acquired by observing a change in vesicles after SNU475 cells were treated with cyclopiazonic acid (CPA) or tunicamycin (TC):

FIG. 8A illustrates ODT images showing the change in vesicles upon CPA treatment; and FIG. 8B illustrates ODT images showing the change in vesicles according to TC treatment.

As illustrated in FIG. 8, it was confirmed that, upon treatment with CPA and TC, the same changes in the vesicles as those upon treatment with thapsigargin occurred. These results indicate that, similarly to the case of thapsigargin, autophagy modulators can be effectively screened through the physical quantities of organelles, which were extracted using the characteristics of vesicles produced in cells and the refractive index distribution of organelles in the cells.

1. A method of screening for an autophagy modulator, the method comprising:
bringing a biological sample into contact with a candidate substance;
measuring refractive index (RI) distribution of an organelle in the biological sample in contact with the candidate substance; and
extracting a physical quantity of the organelle by using the measured RI distribution.

2. The method of claim 1, wherein the biological sample is a cell.

3. The method of claim 2, wherein the method comprises measuring the physical quantity of the organelle in the cell in contact with the candidate substance over time.

4. The method of claim 1, wherein the physical quantity is at least one of a volume of the organelle, a density of the organelle, and a number of the organelles.

5. The method of claim 4, wherein the autophagy modulator is an autophagy activator or an autophagy inhibitor.

6. The method of claim 5, wherein the organelle is any one or more selected from the group consisting of an endoplasmic reticulum (ER), a vesicle, a lipid droplet, a mitochondrion, a ribosome, a Golgi apparatus, an endosome, a nucleolus, and a lysosome.

7. The method of claim 1, wherein the measurement of the RI distribution comprises: generating a plurality of two-dimensional holograms by using an interferometer-based quantitative phase imaging (QPI); and measuring three-dimensional RI distribution by analyzing the plurality of two-dimensional holograms.

8. The method of claim 7, wherein the interferometer-based QPI is optical diffraction tomography (ODT).

9. The method of claim 7, wherein the method comprises generating an image for extracting the physical quantity of the organelle in a cell by using the three-dimensional RI distribution.

10. The method of claim 6, wherein the organelle is a vesicle, and the method comprises determining that, when the volume of the vesicle increases over time, the number of the vesicles decreases over time, or the density of the vesicle decreases over time, the candidate substance is an autophagy activator.

11. The method of claim 6, wherein the organelle is a vesicle, and the method comprises determining that, when the volume of the vesicle does not change or decreases over time, the number of the vesicles does not change or increases over time, or the density of the vesicle does not change or increases over time, the candidate substance is an autophagy inhibitor.

12. The method of claim 1, wherein the method comprises comparing the extracted physical quantity of the organelle with a physical quantity of an organelle in a biological sample not in contact with the candidate substance.

13. The method of claim 12, wherein the physical quantity of the organelle in the biological sample in contact with the candidate substance is a physical quantity extracted immediately after being brought into contact with the candidate substance.

14. The method of claim 13, wherein the method comprises determining that, when a volume of a vesicle in a cell immediately after being brought into contact with the candidate substance increases compared with a volume of a vesicle in a cell not in contact with the candidate substance, when a density of a vesicle in a cell immediately after being brought into contact with the candidate substance increases compared with a density of a vesicle in a cell not in contact with the candidate substance, or when a number of vesicles in a cell immediately after being brought into contact with the candidate substance increases compared with a number of vesicles in a cell not in contact with the candidate substance, the candidate substance is an autophagy activator.

15. The method of claim 13, wherein the method comprises determining that, when a volume of a vesicle in a cell immediately after being brought into contact with the candidate substance does not change or decreases compared with a volume of a vesicle in a cell not in contact with the candidate substance, when a density of a vesicle in a cell immediately after being brought into contact with the candidate substance does not change or decreases compared with a density of a vesicle in a cell not in contact with the candidate substance, or when a number of vesicles in a cell immediately after being brought into contact with the candidate substance does not change or decreases compared with a number of vesicles in a cell not in contact with the candidate substance, the candidate substance is an autophagy inhibitor.

16. The method of claim 10, wherein the vesicle comprises an endoplasmic reticulum.

17. The method of claim 1, wherein the autophagy modulator is used for the prevention or treatment of cancer, a neurodegenerative disease, an autoimmune disease, a cardiovascular disease, a metabolic disease, or a hereditary muscle disease.
